(19) United States
(12) Patent Application Publication
(10) Pub. No.: US 2012/0017902 A1
(43) Pub. Date: Jan. 26, 2012

(54) ELASTOMERIC DISCHARGE MEMBER FOR NASAL DELIVERY DEVICE

(75) Inventors: Robert L. Berger, Bound Brook, NJ (US); Michael L. Turini, Cranford, NJ (US); Peter A. Basile, Bloomsburg, NJ (US); Stephen Miggels, Wyckoff, NJ (US); Henry J. Mack, JR., phillipsburg, NJ (US)

(73) Assignee: Schering Corporation, Kenilworth, NJ (US)

(21) Appl. No.: 13/127,413

(22) PCT Filed: Nov. 3, 2009
(86) PCT No.: PCT/US09/63087

§ 371 Date: Oct. 11, 2011

Related U.S. Application Data
(60) Provisional application No. 61/110,894, filed on Nov. 3, 2008.

(51) Int. Cl. A61M 15/00

(52) U.S. Cl. 128/203.15; 128/203.12

(57) ABSTRACT

Various embodiments of the present invention provide a delivery device (10) for the delivery of a dose of liquid into a nasal cavity. The device includes a discharge member (12) formed for insertion into a nasal cavity, a discharge aperture (14) being formed there through. An elastomeric surface (26) is disposed about the discharge aperture (14).

### FIELD OF THE INVENTION

This invention relates to nasal delivery devices and, more particularly, to discharge members for nasal delivery devices.

### BACKGROUND

Delivery devices for the delivery of doses of liquid into a nasal cavity are well known in the art. In general, these delivery devices include a rigid discharge member which is formed for insertion into the nasal cavity of a user. The discharge member is formed of a rigid plastic which has no forgiveness. Due to the difference in shape of individual nasal cavities and the rigidity of the discharge member, some discomfort may be experienced during usage of these nasal delivery devices.

Additionally, a rigid plastic discharge member will not form a tight seal with a cap used to cover the delivery device. As a result, the nasal spray device typically will incur some evaporation of the liquid compositions contained therein. This evaporation may cause the nasal spray to lose its prime and as such cause a low spray dose emission the next time the nasal spray device is actuated. Some nasal spray devices have incorporated elastomeric components within their caps to form a seal with the nasal spray device cap so that less liquid material will evaporate from the nasal spray container.

Thus, it would be desirable to provide a nasal spray delivery device with a more comfortable discharge member and a discharge member that helps prevent evaporation from the discharge member and loss of prime of the nasal spray device.

### SUMMARY OF THE INVENTION

Several embodiments of the present invention provide for a delivery device for the delivery of at least one dose of a composition that includes a discharge member formed for insertion into a nasal cavity with a discharge aperture being formed there through. An elastomeric surface may be disposed about the discharge aperture. Advantageously, a discharge member may be provided for a nasal delivery device which provides forgiveness in not only providing comfort to a user and also may be capable of forming a vapor tight seal with a cap that covers the discharge aperture.

Several embodiments of the present invention provide a delivery device for the delivery of at least one dose of a composition into a nasal cavity, the device comprising a discharge member formed for insertion into a nasal cavity, a discharge aperture being formed therethrough, wherein an elastomeric surface is disposed about the discharge aperture; a body containing a reservoir of at least one dose. A fluid flowpath may be defined between the reservoir and the discharge aperture, the fluid flowpath being defined by a rigid member in proximity to the discharge aperture.

At least one open void may be defined beneath the elastomeric surface so as to permit deformation of the surface. The elastomeric surface may be formed of a material having a Shore A hardness in the range from about 30 to about 90 or from about 30 to about 60. The elastomeric surface may be formed of a thermoplastic elastomer. A cap may be formed to define a vapor-tight seal about the discharge aperture. The cap may be connected to the body and can be include an elastomeric surface formed to engage the elastomeric surface disposed on the discharge member. The cap may be wholly formed of an elastomeric material. The cap may be removably mountable to the body, the cap encompassing the discharge aperture with the cap being mounted to the body. The cap may be attached or separate from the body. The composition may be a liquid or a solid. A pump or a metered dose canister may be included in the device.

Various other embodiments provide a delivery device for the delivery of at least one dose of a composition into a nasal cavity, the device comprising a discharge member formed for insertion into a nasal cavity, a discharge aperture being formed herethrough, wherein an elastomeric surface is disposed about the discharge aperture; a body containing a reservoir of at least one dose; and, a cap formed to define a vapor-tight seal about the discharge aperture.

Still other embodiments provide for a delivery device for the delivery of at least one dose of a composition into a nasal cavity, the device comprising a discharge member formed for insertion into a nasal cavity, a discharge aperture being formed therethrough, wherein an elastomeric surface is disposed about the discharge aperture; a body containing a reservoir of at least one dose; a cap formed to define a vapor-tight seal about the discharge aperture; and, a pump for urging the at least one dose from the reservoir and through the discharge aperture.

These and other features of the invention shall be better understood through a study of the following detailed description and accompanying drawings.

### BRIEF DESCRIPTION OF THE DRAWINGS:

FIGS. 1-2 show a nasal delivery device having a discharge member and a cap formed in accordance with the subject invention;

FIG. 3 is a partial cross-sectional view taken along line 3-3 of FIG. 2;

FIG. 4 is a partial cross-sectional view taken along line 4-4 of FIG. 2;

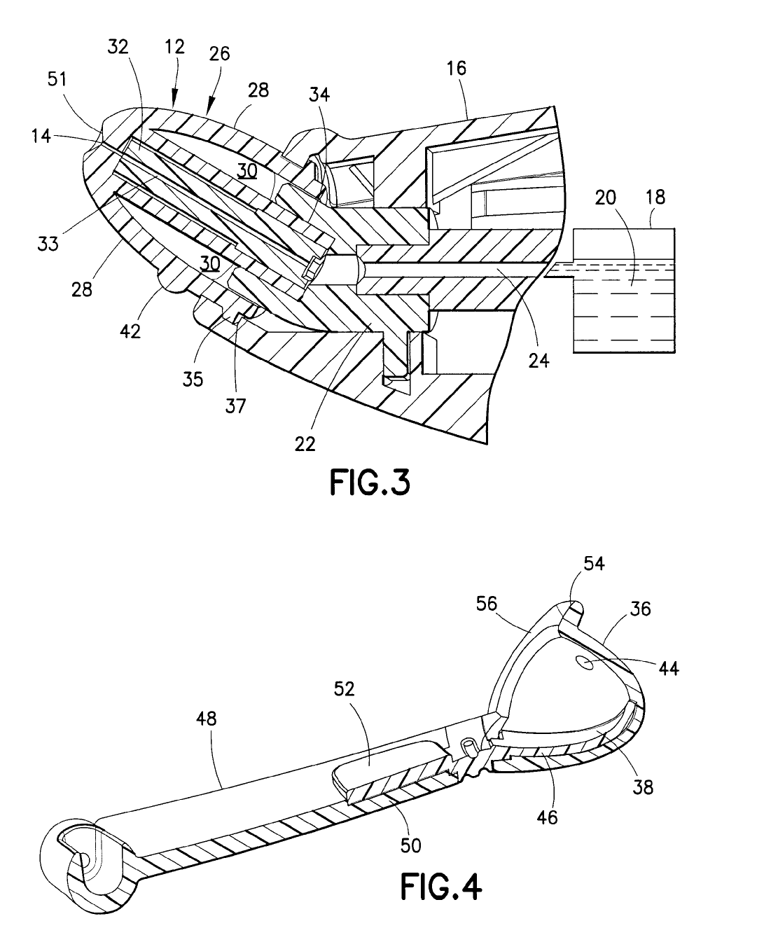

FIG. 3A is a similar view as FIG. 3 but showing a different configuration;

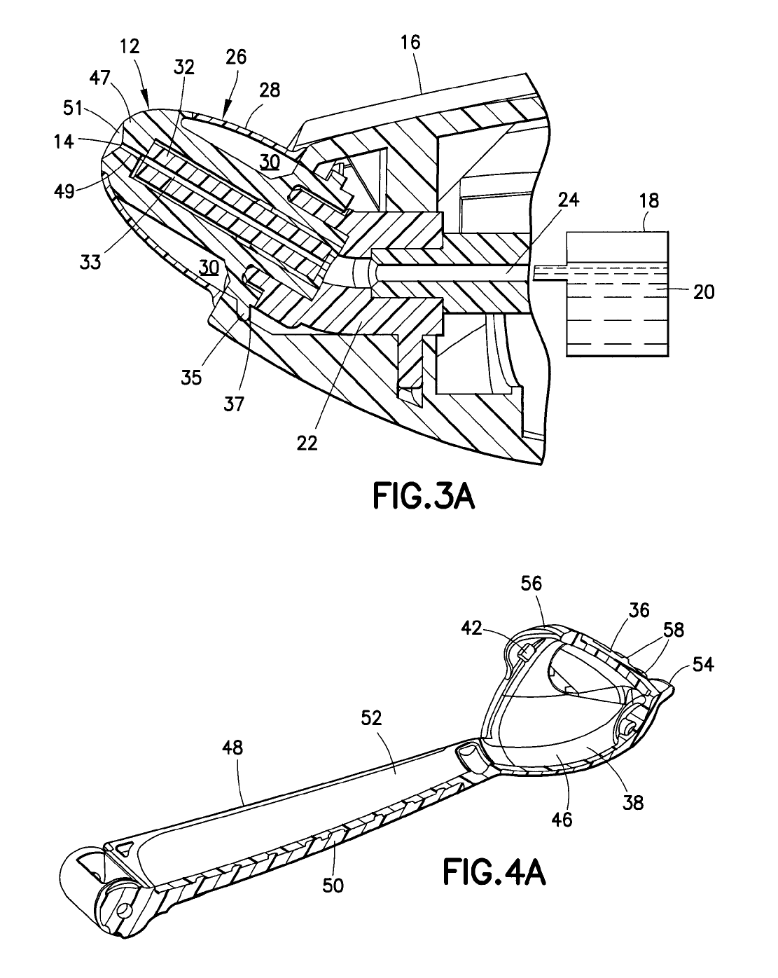

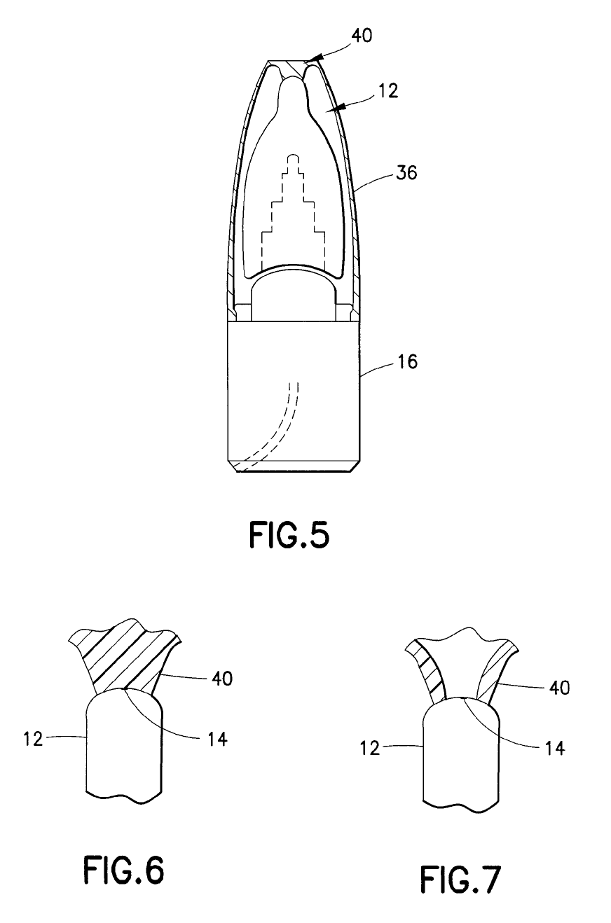

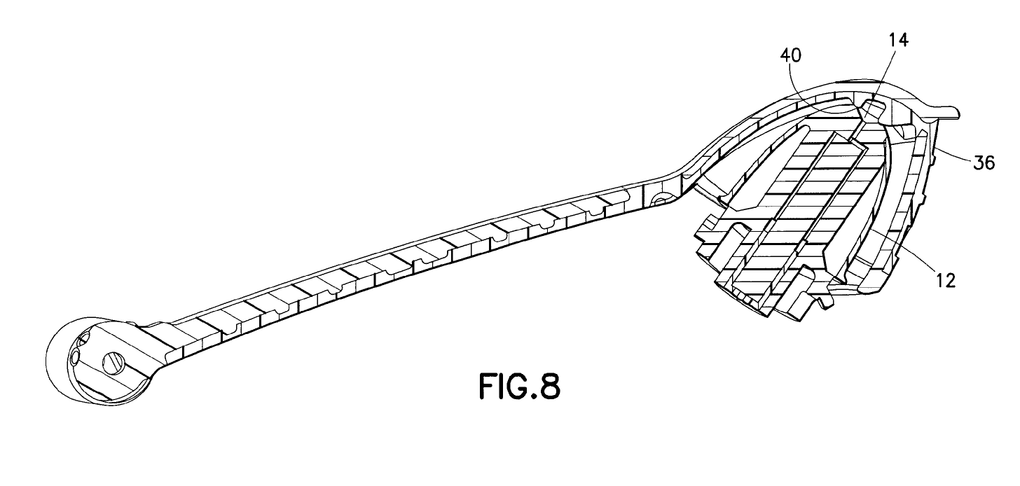

FIG. 4A is a similar view as FIG. 4 but showing a different configuration;

FIG. 5 shows a nasal delivery device having a discharge member and a cap formed in accordance with the subject invention; and,

FIGS. 6-8 show different sealing arrangements usable about the discharge aperture of the discharge member.

### DETAILED DESCRIPTION OF THE INVENTION:

With reference to the figures, a delivery device 10 is shown formed for the delivery of at least one dose into the nasal cavity of a user. The delivery device 10 includes a discharge member 12, having a discharge aperture 14 formed therethrough; a body 16 containing a reservoir 18 formed to accommodate a composition, such as a liquid or a powder. A pump 22 can be configured to urge the at least one dose of the liquid or powder from the reservoir 18 and through the discharge aperture 14.

As will be recognized by those skilled in the art, any configuration of a pump 22 usable to urge the composition 20 from the reservoir 18 and through the discharge aperture 14 may be used. A fluid flowpath 24 is defined between the discharge aperture 14 and the reservoir 18. The pump 22 is configured to urge the composition along the fluid flowpath 24. In addition, as is well known in the art, a pump 22 may be calibrated to meter specific quantities of the composition in defining a dose amount.

A metered dose canister with a propellant such as HFA 227 or HFA 134 may also be utilized.

The composition may be any liquid or powder. With administration into the nasal cavity, one or more pharmaceutically-active agents may be provided in the composition.

The discharge member 12 is shaped to be inserted into the nasal cavity of a user. As shown in the figures, the discharge member 12 may have an elongated shape and may be tapered or rounded towards the discharge aperture 14. The discharge member 12 may have a length from about 0.375″ to about 0.75″ and a maximum diameter of about 1 inch. With the configuration shown in the figures, the discharge member 12 generally has a bullet shape. The discharge aperture 14 may be formed at the center and at the furthestmost extent of the discharge member 12.

An elastomeric surface 26 is disposed on the discharge member 12 about the discharge aperture 14. The elastomeric surface 26 may be defined by a layer of elastomeric material which has no backing or, alternatively, is provided with backing. The elastomeric surface 26 may be provided with a sufficient level of forgiveness to provide a user with comfort during use. The thickness, durometer and/or rigidity of any backing that is used may be adjusted to provide the elastomeric surface 26 with a desired level of forgiveness, i.e., compressibility.

With reference to FIG. 3, the fluid flowpath 24 travels through the discharge member 12 to reach the discharge aperture 14. A sidewall portion 28 of the discharge member 12 encircles the fluid flowpath 24. The discharge member 12 may be configured to have forgiveness such that the sidewall portion 28 has compliance in a radially inward direction. In this manner, the discharge member 12 may be compliantly deformed in response to the diameter of a nasal cavity. To enhance this compliance, one or more open voids 30 may be defined interiorly of the sidewall portion 28. A single of the voids 30 may be provided which is annular and encircles the fluid flowpath 24. The sidewall portion 28 may be wholly formed of an elastomeric material and defines the elastomeric surface 26. The voids 30 are defined interiorly of the sidewall portion 28 so that the sidewall portion 28 may deflect inwardly. As shown in FIG. 3, the elastomeric surface 26 may extend about the entire surface of the discharge member 12. To provide stability to the elastomeric surface 26, a rigid member 32 (e.g., formed of thermoplastic), having a passage 33 therethrough which defines a portion of the fluid flowpath 24, may be disposed adjacent to the discharge aperture 14. An inner flange 34 may be defined on the interior of the discharge member 12 formed to engage the rigid member 32 in generating a holding force for retaining the discharge member 12 on the rigid member 32. The inner flange 34 may be integrally formed with the elastomeric surface 26, with all portions thereof being formed of an elastomeric material.

A rigid terminal member 47 (e.g., formed of thermoplastic), having a passage 49 formed therethrough, may define the discharge aperture 14, as shown in FIG. 3A. The terminal member 47 may include a pocket 51 defined at the terminus of the passage 49. The rigid member 32 may be seated in the terminal member 47 so as to have the passage 33 in communication with the passage 49. The rigid member 32 need not be utilized with the fluid flowpath 24 extending to the passage 49. The sidewall portion 28 may be provided directly about the terminal member 47 or about one or more of the voids 30 located about the terminal member 47. The terminal member 47 may be desired where concerns exist over leaching or other residual effects of the elastomeric material of the sidewall portion 28 on liquid or powder to be administered by the dispenser 10 and remnants thereof located about the discharge aperture 14. A more impervious surface about the discharge aperture 14 may be defined by the terminal member 47, as opposed to the elastomeric material.

The discharge member 12 may be formed integrally with the body 12 or mounted thereto. As shown in FIGS. 3 and 3A, a mounting flange 35 may be provided on the discharge member 12 formed to be received in a channel 37 formed in the body 16. The reverse of this configuration, as well as other mounting configurations, may be utilized.

Suitable elastomeric materials include a thermoplastic elastomer, such as that sold under the trademark “SANTOPRENE” by Exxon Mobil Corporation. Further Suitable elastomeric materials has a durometer in the range from about 30 to about 90 Shore A hardness, or in the range from about 30 to about 60 Shore A hardness. In addition, the elastomeric material is preferably free of components that can disperse into the liquid 20 upon delivery of a dose of the liquid 20 from the discharge aperture 14. The elastomeric material may be any rubber (organic or inorganic) or rubber copolymer, and may be selected from the following, taken alone or in any combination: thermoplastic elastomer; ethylene-propylene-diene monomer (EPDM); silicone based rubber; random or block styrene-butadiene copolymer, such as styrene-butadiene rubber and high styrene rubber; isoprene rubber, including random or block styrene-isoprene rubber; chloroprene rubber; saturated polyolefin rubber; acrylonitrile-butadiene copolymers; epichlorohydrin rubber; propylene oxible rubber; ethylene-acrylic rubber; and, norbornene-based elastomers or combinations thereof.

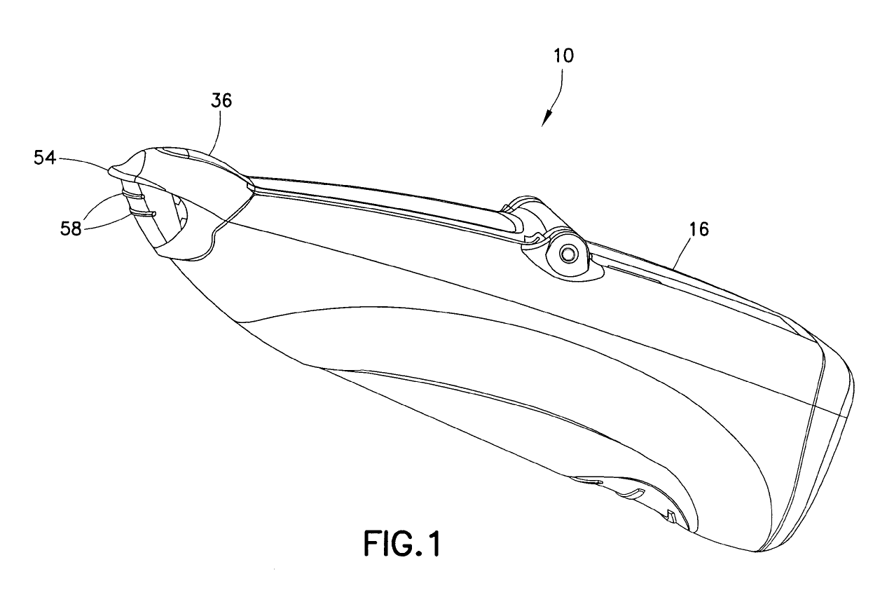

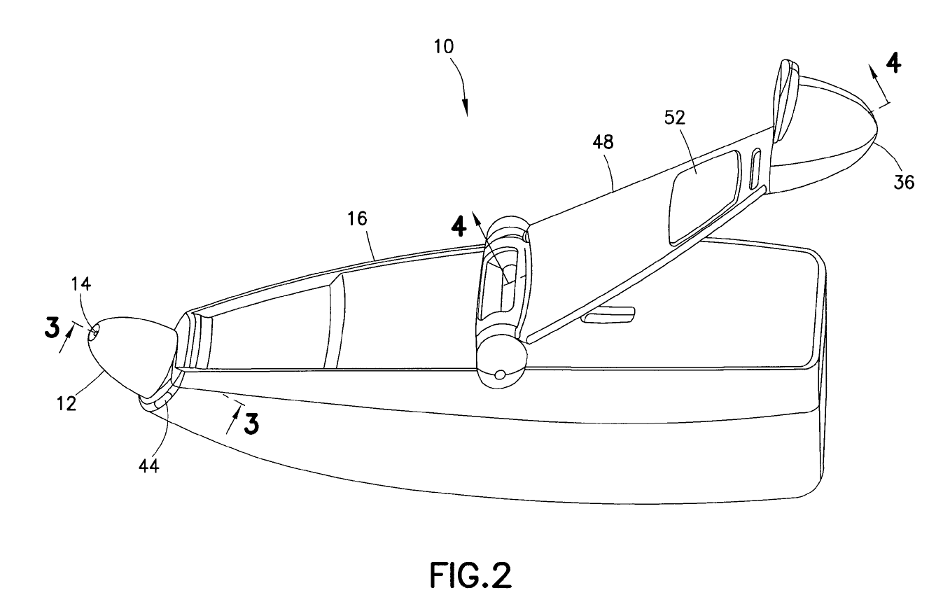

With reference to FIGS. 1, 5 and 8, a cap 36 may be provided for covering, and encompassing, the discharge aperture 14 during non-use. The cap 36 may be configured to define a vapor-tight seal about the discharge aperture 14 to try to prevent evaporative leakage, which not only leads to loss of the liquid 20 but also to loss of priming. A vapor tight seal is defined as a seal that substantially minimizes or prevents vapor transmission. A vapor tight seal will minimize or prevent a loss of prime due to evaporative loss of the nasal spray device over a period of at least about 30 days, at least about 45 days, at least about 60 days, at least about 90 days or at least about 180 days or longer. The seal may be defined using known techniques. For example, inner surface 38 of the cap 36 may be formed to sufficiently tightly engage the discharge member 12 in a continuous perimeter about the discharge aperture 14 so as to define the seal. With reference to FIG. 5, a seal protrusion 40 may extend from the inner surface 38 to engage the discharge member 12. With reference to FIG. 6, the seal protrusion 40 may be formed at least partially solid to span across the discharge aperture 14 continuously in defining the seal. Alternatively, as shown in FIGS. 7 and 8, the seal protrusion 40 may have portions which collectively define a continuous sealing perimeter which bounds about the discharge aperture 14. To ensure that the seal is properly maintained, the cap 36 may be formed to be tightly, yet releasably, mounted to the body 16. Known configurations for the releasable mounting may be utilized, including cooperating detents/wells, interference fits and/or deflectable or adjustable elements. For example, with reference to FIG. 3, a detent 42 may be defined on the discharge member 12 which is formed to be received in a well 44 defined on the inner surface 38 of the cap 36. Alternatively, as shown in FIG. 4A, one or more of the detents 42 may be formed on the cap 36 to engage one of more of the wells 44 defined on the body 16 (FIG. 2) and vice versa in varying combinations. A releasable mounting arrangement can be used that does not have features located on the discharge member 12.

All or a portion of the cap 36 may be also formed from an elastomeric material. The cap 36 may be formed at least partially rigid, particularly where the releasable mounting elements are located, to ensure minimal deformation after multiple uses. With reference to FIGS. 4 and 4A, an elastomeric liner 46 may be disposed over all or a portion of the inner surface 38. The elastomeric liner 46 may be located to span across the discharge aperture 14. Thus, with the cap 36 mounted to the body 16, the elastomeric surface 26 and the elastomeric liner 46 together define a seal.

The cap 36, for example, as shown in FIG. 5, may be formed to be completely separated (unattached) from the body 16. Alternatively, as shown in FIGS. 1 and 2, the cap 36 may be connected to the body 16 such as via connecting arm or tether 48. For comfort during use, one or more sections 50 of the connecting arm 48 and/or the body 16 may be provided with an elastomeric covering 52. The elastomeric material used in conjunction with the cap 36, the body 16 and/or the connecting arm 48 may be the same as that specified above with respect to the discharge member 12.

In addition, a finger rest 54 may protrude from the cap 36 configured to facilitate removal of the cap 36 from the discharge member 12 during use. As shown in FIG. 4A, the finger rest 54 is located spaced from bottom edge 56 of the cap 36. This keeps a user's fingers away from the discharge member 12. As shown in FIG. 4, the finger rest 54 may be located in proximity to the bottom edge 56. In addition, one or more friction-enhancing ribs 58 may be defined on the cap 36 to enhance frictional engagement therewith.

The descriptions of the embodiments of the invention have been presented for purpose of illustration and description. They are not intended to be exhaustive or to limit the invention to the precise forms disclosed, and obviously many modifications and variations are possible in light of the above teaching.

What is claimed is:

1. A delivery device for the delivery of at least one dose of a composition into a nasal cavity, the device comprising:
a discharge member formed for insertion into a nasal cavity, a discharge aperture being formed therethrough, wherein an elastomeric surface is disposed about the discharge aperture;
a body containing a reservoir of at least one dose.

2. A delivery device as in claim 1, wherein a fluid flowpath is defined between the reservoir and the discharge aperture, the fluid flowpath being defined by a rigid member in proximity to the discharge aperture.

3. A delivery device as in claim 1, wherein at least one open void is defined beneath the elastomeric surface so as to permit deformation of the surface.

4. A delivery device as in claim 1, wherein the elastomeric surface is formed of a material having a Shore A hardness in the range from about 30 to about 90.

5. A delivery device as in claim 4, wherein the Shore A hardness is in the range from about 30 to about 60.

6. A delivery device as in claim 1, wherein the elastomeric surface is formed of a thermoplastic elastomer.

7. A delivery device as in claim 1, further comprising a cap formed to define a vapor-tight seal about the discharge aperture.

8. A delivery device as in claim 7, wherein the cap is connected to the body.

9. A delivery device as in claim 7, wherein the cap includes an elastomeric surface formed to engage the elastomeric surface disposed on the discharge member.

10. A delivery device as in claim 7, wherein the cap is wholly formed of an elastomeric material.

11. A delivery device as in claim 7, wherein the cap is removably mountable to the body, the cap encompassing the discharge aperture with the cap being mounted to the body.

12. A delivery device as in claim 7, wherein the cap is separate from the body.

13. A delivery device as in claim 1, wherein the composition is a liquid.

14. A delivery device as in claim 1, wherein the composition is a powder.

15. A delivery device as in claim 1, wherein the elastomeric surface is defined from a material selected from the group consisting of: thermoplastic elastomer; ethylene-propylene-diene monomer (EPDM); silicone based rubber; random or block styrene-butadiene copolymer, such as styrene-butadiene rubber and high styrene rubber; isoprene rubber, including random or block styrene-isoprene rubber; chloroprene rubber; saturated polyolefin rubber; acrylonitrile-butadiene copolymers; epichlorohydrin rubber; propylene oxible rubber; ethylene-acrylic rubber; norbornene-based elastomers, and combinations thereof.

16. A delivery device for the delivery of at least one dose of a composition into a nasal cavity, the device comprising:
a discharge member formed for insertion into a nasal cavity, a discharge aperture being formed therethrough, wherein an elastomeric surface is disposed about the discharge aperture;
a body containing a reservoir of at least one dose; and,
a cap formed to define a vapor-tight seal about the discharge aperture.

17. A delivery device as in claim 16, wherein the cap includes an elastomeric surface formed to engage the elastomeric surface disposed on the discharge member.

18. A delivery device as in claim 16, wherein the cap is removably mountable to the body, the cap encompassing the discharge aperture with the cap being mounted to the body.

19. A delivery device as in claim 16, wherein the cap is connected to the body.

20. A delivery device for the delivery of at least one dose of a composition into a nasal cavity, the device comprising:
a discharge member formed for insertion into a nasal cavity, a discharge aperture being formed therethrough, wherein an elastomeric surface is disposed about the discharge aperture;
a body containing a reservoir of at least one dose;
a cap formed to define a vapor-tight seal about the discharge aperture; and,
a pump for urging the at least one dose from the reservoir and through the discharge aperture.
